(19) United States
(12) Patent Application Publication
(10) Pub. No.: US 2022/0401752 A1
(43) Pub. Date: Dec. 22, 2022

(54) WOUND CARE METHOD, SYSTEM, AND DEVICES

(71) Applicant: Lumen Catheters, LLC, Marlborough, MA (US)

(72) Inventor: Adam E.M. Eltorai, Marlborough, MA (US)

(21) Appl. No.: 17/851,572

(22) Filed: Jun. 28, 2022

Related U.S. Application Data
(63) Continuation-in-part of application No. 17/665,965, filed on Feb. 7, 2022.
(60) Provisional application No. 63/146,697, filed on Feb. 7, 2021.
(60) Provisional application No. 63/215,910, filed on Jun. 28, 2021.

(51) Int. Cl. A61N 5/06

(52) U.S. Cl. CPC A61N 5/0624; A61N 5/0616; A61N 2005/0645; A61N 2005/0652; A61N 2005/0663; A61N 2005/0661

(57) ABSTRACT

A device and method for disinfecting and/or enhancing healing of a wound or surgical site facilitates placing a light-emitting device with a frame having a central void, the central void adapted to surround the wound or surgical site; an illumination source disposed on the frame for directing safe, therapeutic light centrally towards the underlying wound or surgical site; and the transparent central void enabling visualization and clinical monitoring of the underlying wound or surgical site.

### RELATED APPLICATIONS

This Patent Application claims the benefit under 35 U.S.C. § 119(e) of U.S. Provisional Patent App. No. 63,215,910, filed Jun. 28, 2021, entitled “ANTIMICROBIAL LIGHT-EMITTING DEVICES,” and is a Continuation-in-Part (CIP) of U.S. patent application Ser. No. 17/665,965, filed Feb. 7, 2022, entitled “ANTIMICROBIAL LIGHT-EMITTING PERCUTANEOUS SITE DRESSING,” which claims the benefit under 35 U.S.C. § 119(e) of U.S. Provisional Patent App. No. 63,146,697, filed Feb. 7, 2021, entitled “ANTIMICROBIAL LIGHT DRESSING DEVICE,” incorporated herein by reference in entirety.

### BACKGROUND

Healthcare providers often access treatment areas through the use of elongated devices that penetrate or pierce a physiological boundary, such as the skin/epidermal system, gastrointestinal, urinary, nasal and ocular, to name several. The penetration of a foreign member introduces a risk of adverse results from infection resulting from the artificial path created by the inserted foreign member. Particularly in a healthcare environment, where many therapeutic procedures utilize these foreign members, the risk of provider or hospital caused infections is prevalent.

A wound is a disruption of the normal structure and function of the skin and soft tissue architecture. An acute wound demonstrates normal physiology, and healing is anticipated to progress through the expected stages of wound healing, whereas a chronic wound is broadly defined as one that is physiologically impaired.

To ensure proper healing through the expected stages, the wound base should be well vascularized, free of devitalized tissue, clear of infection, and moist. Wound dressings might help facilitate this process if they eliminate dead space, control exudate, prevent bacterial overgrowth, ensure proper fluid balance, demonstrate cost-efficiency, and are manageable for the patient and/or nursing staff. Wounds with progressive healing as evidenced by granulation tissue and epithelialization can undergo closure or coverage. All wounds are colonized with microbes; however, not all wounds are infected. Wound infections can lead to morbidity and mortality.

Surgical site infections (SSIs) are a common cause of health care-associated infection. SSIs are often localized to the incision site (superficial/deep incisional SSI) but can also extend into deep tissues. Careful infection control and efforts to reduce skin flora burden are essential to reduce morbidity and mortality.

### SUMMARY

Configurations herein are based, in part, on the observation that therapeutic procedures and treatment often involve a foreign member for transfer of fluids or samples between the human patient body and a treatment source or testing facility. Unfortunately, conventional approaches often involve the use of a foreign member such as a needle, vessel or probe to cross an external bodily boundary to access various organ systems for used in patient care. Insertion or breach into the bodily region by these foreign members can form a path for pathogens such as bacteria and other microorganisms to cause infection. Whether the foreign member remains for an extended period, or is withdrawn and leaves a wound, a path of vulnerability is exposed for pathogens. Accordingly, configurations herein substantially overcome the shortcomings of the infection risk presented by conventional foreign members by providing an antibacterial, antipathogen light source for illuminating or irradiating a treatment region defining an insertion point of epidermal, gastrointestinal, urinary, or oral breach by a foreign member used in the course of treatment.

Catheter usage for urinary tract intervention involves insertion of a catheter vessel for urethral engagement. An inserted catheter vessel or tube, even if sterile upon insertion, presents a path for pathogens into the urethra and bladder. A circumferential frame having an array of lights around a perimeter and directed for focusing on a central void for illuminating a catheter provides a barrier to passage of pathogens. The frame provides an antimicrobial and safe light emission having a wavelength of or around 222 nm far UVC or 405 nm visible blue light for eradicating any bacteria or pathogens prior to infiltration via the catheter vessel.

In the case of percutaneous breach, intravenous delivery of medication is an effective medium for medicinal treatment directly to blood or tissue, which allows the medication to be quickly delivered to a specific region. General bloodstream delivery avoids degradation that can occur by oral administration which must pass via the gastrointestinal barrier. Unfortunately, conventional approaches to percutaneous delivery, typically via a needle or similar insertion member, suffer from the shortcoming that they pose an infection risk from a breach of the natural dermal (skin) barrier which guards against infiltration of pathogens. Typically, an antimicrobial substance is applied around the insertion point of the needle, however such chemical based approaches generally have diminishing effects over time, and need repeated applications for continued effectiveness. Accordingly, configurations herein substantially overcome the shortcomings of chemical and topical approaches by providing an antimicrobial light dressing device, system and method for a percutaneous treatment that bathes a treatment region around the percutaneous insertion with an antibacterial illumination source for preventing pathogens around the insertion from entering via the dermal puncture created by the insertion. The antimicrobial light dressing device combines a circumferential body centered around the insertion, and an arrangement of LEDs around the body that focus the light around the insertion and onto a therapeutic region of the insertion. An opening in the circumferential body has an articulated protrusion for offsetting a medicinal vessel such as an IV tube off the skin surface to avoid blocking light to an area under the vessel. The result is a 360 degree coverage of antimicrobial light around the percutaneous insertion as the medicinal vessel contacts the skin surface only at the insertion point in the center of the treatment region.

### BRIEF DESCRIPTION OF THE DRAWINGS

The foregoing and other objects, features and advantages of the invention will be apparent from the following description of particular embodiments of the invention, as illustrated in the accompanying drawings in which like reference characters refer to the same parts throughout the different views. The drawings are not necessarily to scale, emphasis instead being placed upon illustrating the principles of the invention.

FIG. 1 is a context view of a medical treatment environment suitable for use with configurations herein;

FIG. 2 is a perspective view of the medical device for antimicrobial light treatment of a percutaneous insertion site;

FIG. 2A is a perspective view of the medical device for antimicrobial light treatment of a wound or incision site;

FIGS. 3A-3C show engagement of the device of FIG. 2 with a treatment region defined by the percutaneous insertion site;

FIGS. 4A-4B show perspective views of a central void in the device of FIGS. 1-3c;

FIG. 5 shows a plan view of the device of FIGS. 1-4B;

FIG. 6 shows a side, cutaway view of the central void and illumination cavity formed by the device of FIGS. 1-5;

FIG. 7 shows a perspective view of the illumination cavity of FIG. 6;

FIG. 8 shows a bottom view of the device of FIGS. 1-7;

FIG. 9 shows an underside perspective view of the device and illumination/light cavity of FIGS. 6-8;

FIG. 10 shows a method of applying the antimicrobial light treatment of FIGS. 1-9;

FIG. 11 shows a perspective view of an alternative configuration of the safety device for use with an epidermal incision and/or treatment;

FIG. 12 shows a top view of the device of FIG. 11 engaged with an epidermal treatment region; and

FIG. 13 shows a schematic view of the device of FIGS. 11 and 12 for wound treatment coverage.

### DETAILED DESCRIPTION

A device for the dressing of wounds and insertion sites of percutaneous and drug delivery devices provides circumferential protection of a wound or insertion site of a percutaneous or drug delivery device. In particular, the device is an integrated dressing for vascular and non-vascular percutaneous medical devices (e.g., IV catheters, central venous lines, arterial catheters, dialysis catheters, peripherally inserted coronary catheters, mid-line catheter, drains, chest tubes, externally placed orthopedic pins, ventricular assist device drivelines, and epidural catheters) comprising an adhesive dressing and an antimicrobial light source, such as visible light, far UVC light, and any suitable electromagnetic emission of a therapeutically beneficial wavelength. The dressing device reduces infection risk from vascular and non-vascular percutaneous medical devices by providing sufficient tissue-safe antimicrobial light at a wound or insertion site.

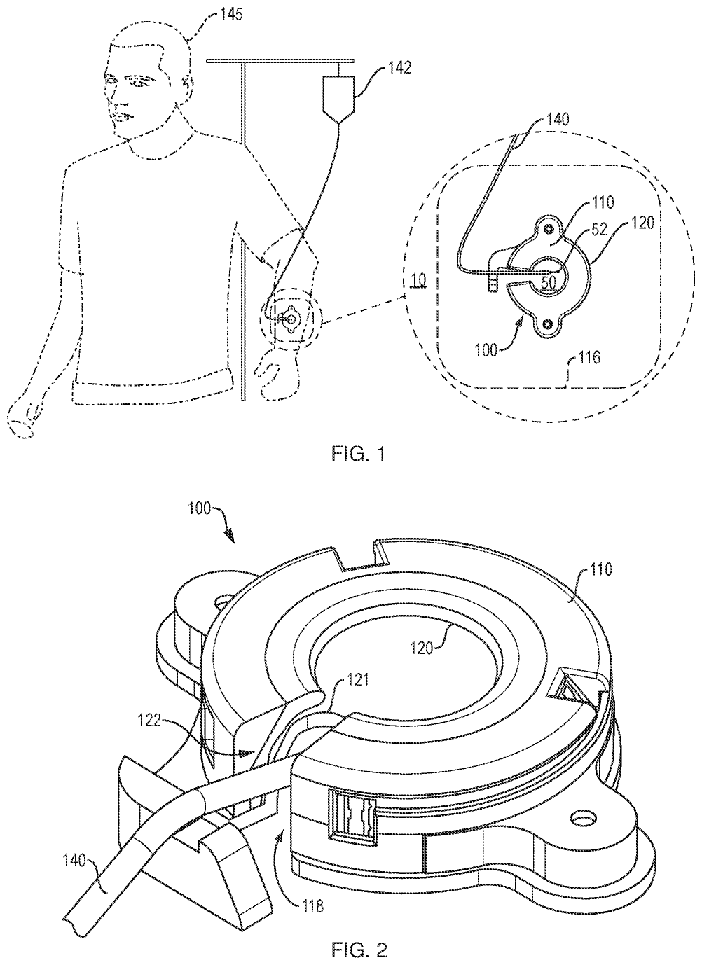

FIG. 1 is a context view of a medical treatment environment suitable for use with configurations herein. Referring to FIG. 1, an antimicrobial epidermal device 100 includes a circumferential light-emitting body 110 configured for adhesion around a percutaneous insertion site 52 for directing therapeutic light at the percutaneous insertion site while permitting unobstructed passage of a medication vessel 140 to the percutaneous insertion site 52. The medication vessel emanates from a fluidic source 142 of medication or other liquid, such as an IV (Intravenous) bag. The percutaneous insertion site 52 defines a surrounding treatment region 50, typically on an arm of a patient 145 because of ease of IV access, however any suitable epidermal region may be selected for the percutaneous insertion site.

In the antimicrobial epidermal device 100, the circumferential body 110 is adapted for epidermal placement on the treatment region 50 of a larger epidermal surface 10. Placement is based on a central void 120 in the circumferential body for epidermal access and alignment generally over the insertion site 52. The circumferential body 110 includes an illumination source disposed for emitting a therapeutic light on the treatment region 50 defined by the central void 120. An adhesive member 116, such as a patch or bandage, adheres the circumferential body 110, vessel 140 and a percutaneous penetration member such as a needle to the epidermal area around the treatment region 50.

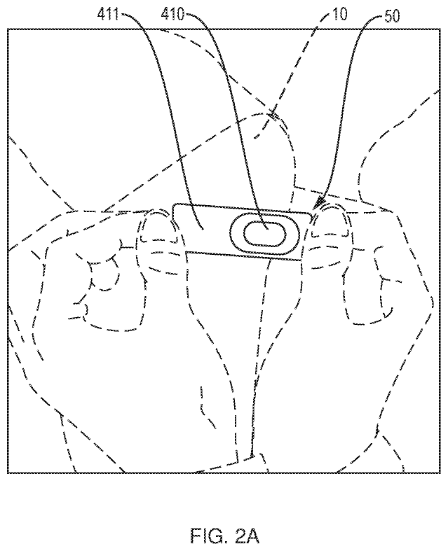

FIG. 2 is a perspective view of the medical device of FIG. 1 for antimicrobial light treatment around a percutaneous insertion site 52. FIG. 2 shows the central void 120 accessible by a vessel gap 122 in the circumferential body 110 for passage of the medication vessel 140 to a penetration or insertion member defining the insertion site 52. The treatment region 50 is defined by a radius around the insertion site roughly centered within the circumferential body. In the example of FIG. 2, the central void 120 remains covered by an insert 121 except at the vessel gap 122 for permitting vessel access into an illumination cavity 118.

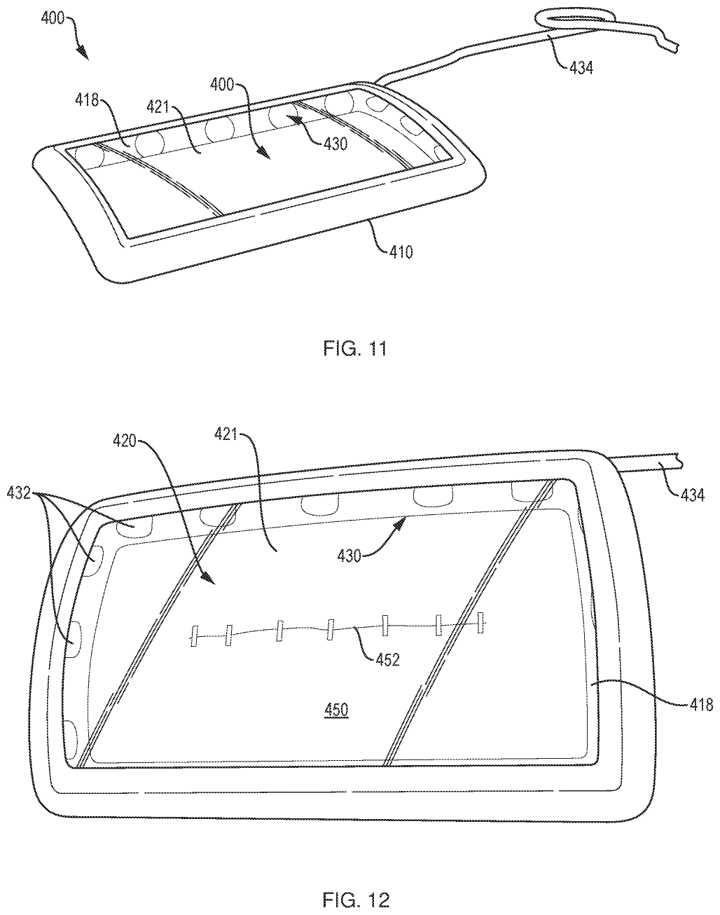

FIG. 2A is a perspective view of the medical device for antimicrobial light treatment of a wound or incision site. In FIG. 2A, the treatment region 50 requires surface based intervention from a therapeutic light in a frame 410 secured by an adhesive patch 411 or bandage, discussed further below in FIGS. 11-15. The light-emitting wound closure safety device includes the frame 410 having a central void adapted to surround a wound or surgical site, and a small LED or illumination source disposed on the frame for directs safe, therapeutic light centrally towards the underlying wound or surgical site.

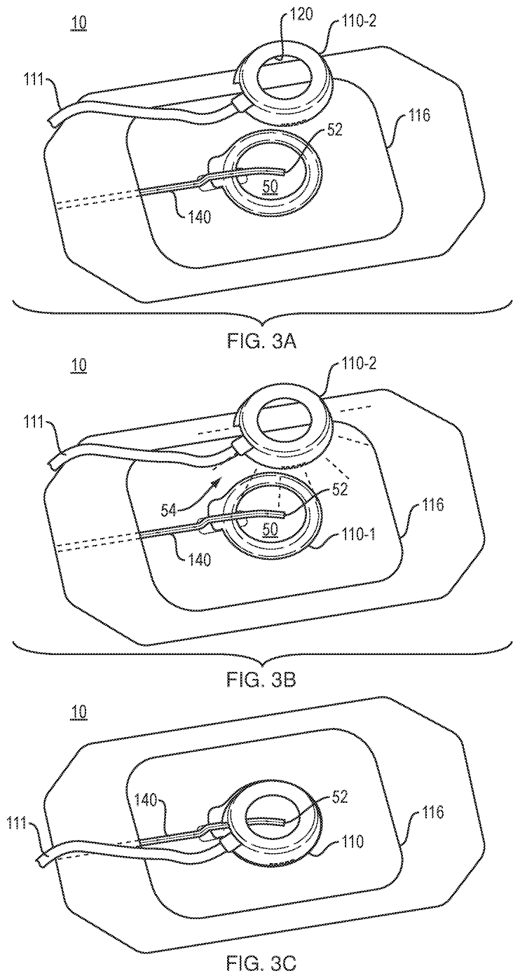

FIGS. 3A-3C show engagement of the device of FIG. 2 with a treatment region defined by the percutaneous insertion site. In a particular configuration, the device may be combined with an adhesive member 116 such as a sheet, patch or bandage for providing a system of secure attachment of the illumination source to the percutaneous insertion site. Referring to FIGS. 1-3C, the circumferential body 110 is disposed on an epidermal surface 10, in conjunction with an adhesive member 116. The adhesive member 116 has an adhesive attraction to the epidermal surface 10 and extends over the treatment region 50 and is disposed for securing the circumferential body 110 and a treatment vessel 140 directed to the central void 120. The adhesive member my include a securement or fixation dressing having adhesive and therapeutic or antimicrobial properties. The securement or fixation dressing is disposed between the circumferential body 110 and the epidermal surface 10. The circumferential body is therefore disposed in place by the underlying securement or fixation dressing/patch, and substantially centered around the insertion site.

The configuration of FIG. 3 shows a two-part configuration of the device. The circumferential body 110 further includes a distal layer 110-2 including a power connection 111 for powering an illumination source such as one or more LED elements and a proximate layer 110-1 having a translucent surface, in which the LED elements are disposed within the distal layer 110-2 for directing the therapeutic light onto the treatment region 50.

The proximate layer 110-1 engages with the adhesive member 116, which may be integrated as an adhesive whole or applied in separate phases. In the configuration of FIGS. 3A-3C, the adhesive member 116 may reside between the proximate layer 110-1 and distal layer 110-2. The adhesive member 116 secures the insertion member at the insertion site 52 alone with the medication vessel 140, shown in FIG. 3A. The treatment region 50 is defined by a radius around the dermal insertion site 52, where the insertion site 52 provides the dermal access for medical intervention through the skin by a sharp, piercing structure.

In FIG. 3B, the distal layer 110-2 approaches the secured, proximate layer 110-1. The distal layer 110-2 may already be emitting light 54 onto the treatment region 50. In FIG. 3C, the distal layer 110-2 engages the proximate portion 110-2 to form the full circumferential member 110, and encapsulates an illumination cavity, discussed further below.

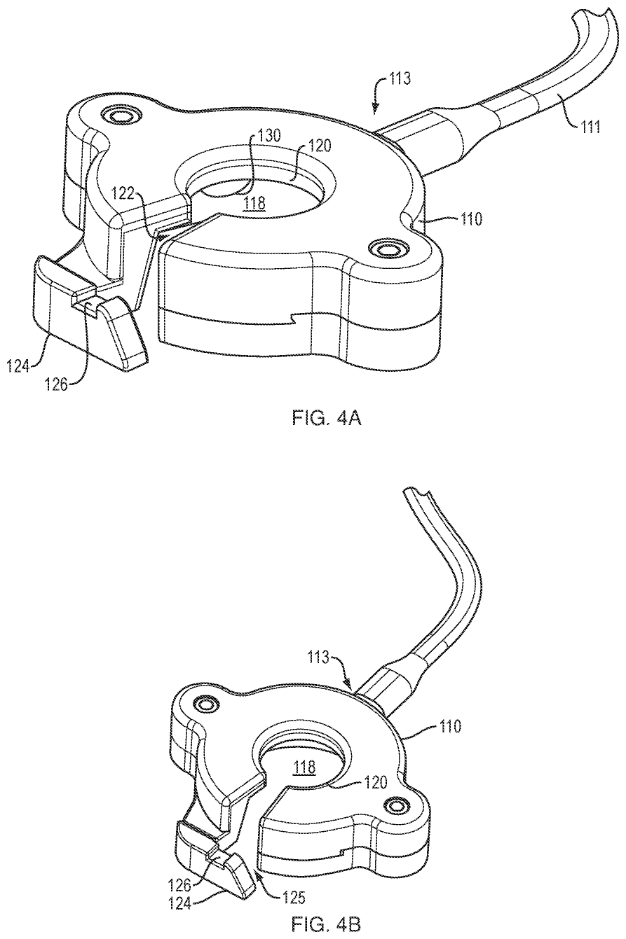

FIGS. 4A-4B show perspective views of a central void 120 in the device of FIGS. 1-3c . Referring to FIGS. 1-4B, upon adherence and proper administration, the circumferential body 110 adheres to the epidermal surface 10 with the central void 120 roughly centered on the insertion site 52. An illumination source 130 includes at least one LED element defining the illumination source, in which the LED element emits a wavelength based on an antimicrobial effect. The central void 120 has a size based on a treatment vessel 140 size and clearance over the insertion site 152. The treatment vessel 140 has an attachment to the insertion member such as a needle for a percutaneous insertion under the central void. The vessel extends through the vessel gap 122 and through the central void 120 or at least through the gap 122 and into the illumination cavity 118.

A power connection 113 receives the power supply 111 on the circumferential body 110. The power supply couples to the illumination source 130 and is adapted for receiving an electrical source for powering the illumination source, such as an external USB, batteries, AC or similar AC or DC source based on the electrical requirements of the illumination source 30. A discontinuity in the circumferential body defines the vessel gap 122 for accommodating the treatment vessel 140. The treatment vessel 140 couples to the percutaneous insertion member in the treatment region 50 under the central void 120. Routing of the treatment vessel 140 is provided by a protrusion 124 extending outward from the circumferential body. The protrusion 124 has an elevated surface 126 disposed away from the epidermal surface 10, such that the elevated surface 126 is adjacent the vessel gap 122 for directing the treatment vessel at an offset distance from the dermal surface 10. Elevation of the treatment vessel 140 above the skin avoids a shadow from the light and instead allows a shadowed region 125 to be reached by light from the illumination source 130 rather than being shaded or obscured by the vessel 140 from reaching the skin at the shadowed region.

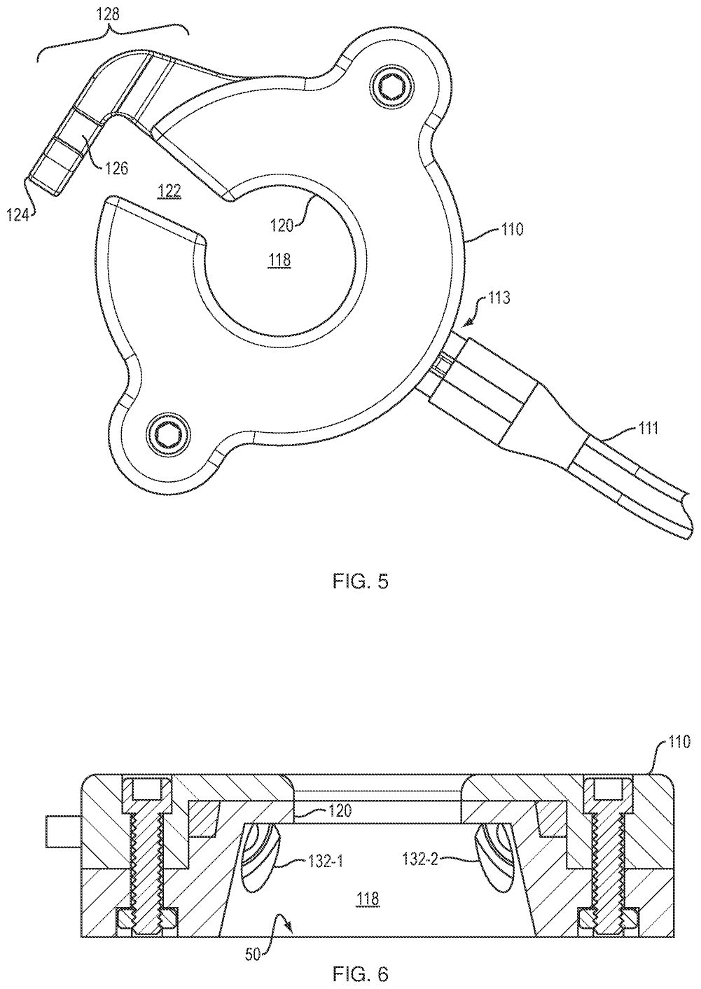

FIG. 5 shows a plan view of the device of FIGS. 1-4B. Referring to FIGS. 1-5, the vessel gap 122 is an opening or passage in the circumferential body 110. A lateral extension 128 extends radially from the circumferential body 110 adjacent the vessel gap 122, and turns toward the gap 122 to provide the elevated surface 126 residing on the protrusion 124. The elevated surface 126 is disposed on a path towards the central void 120 for receiving a treatment vessel 140 disposed on the path for fluidic delivery to an insertion site 52 in the treatment region 50.

FIG. 6 shows a side, cutaway view of the central void and light cavity formed by the device of FIGS. 1-5. Referring to FIGS. 1-6, a plurality of LED elements 132-1 . . . 132-2 (132 generally) surround the illumination cavity 118, although as few as 1 could be provided. In the example configuration, the plurality of LED elements 132 are disposed generally in a circle around the circumferential body 110, and fill the illumination cavity 118 with light focused on the treatment region 50. The inner surface of the circumferential body 110 and optional insert 121 are a light color and may be translucent to reflect and refract (distribute and target) as much if the light as possible around the illumination cavity 118 to fall on the treatment region 50. The antimicrobial light is therefore specifically targeted to fall on the treatment region defined by the percutaneous insertion and surrounding epidermal region, specifically within the illumination cavity 118 of the circumferential body 110.

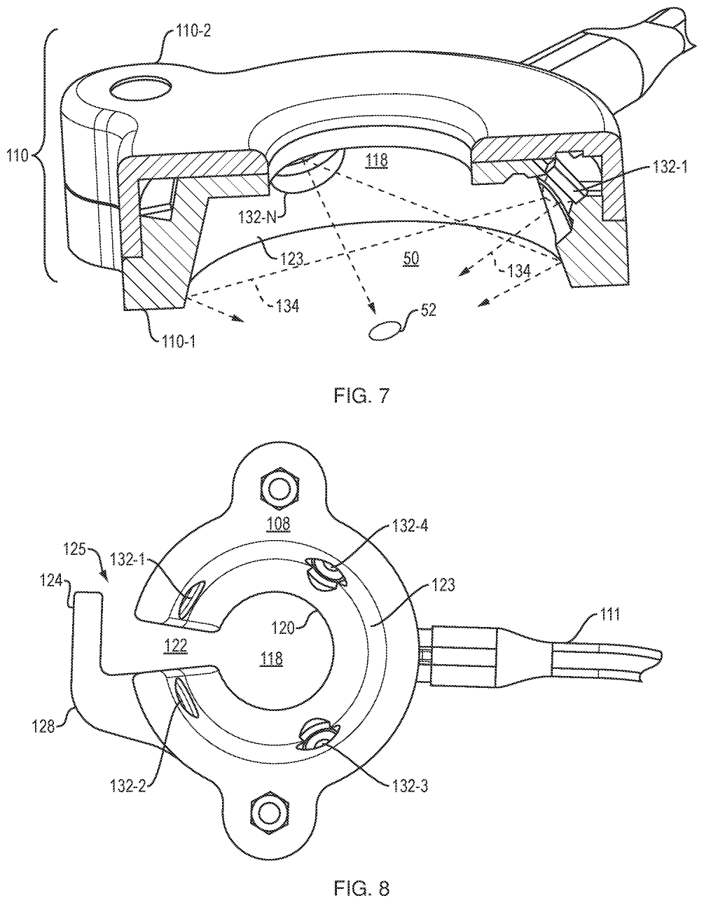

FIG. 7 shows a perspective view of the light cavity of FIG. 6 as a cutaway from the circumferential body 110. Referring to FIGS. 1-7, the circumferential body 110 is disposed on a treatment region 50 and centered on or around an insertion site 52 of a percutaneous insertion member. One or more LED elements 132-N disposed on an inner surface 123 of the circumferential body bathe the illumination cavity 118 in light for directing the light directly on the treatment region 50 and also reflected and/or refracted around the inner surface 123 as shown by arrows 134. A light colored, translucent and/or reflective property of the inner surface 123 generally focuses direct and indirect light onto the treatment region 50 for eliminating harmful pathogens that may live on the skin surface around the insertion site 52.

FIG. 8 shows a bottom view of the device of FIGS. 1-7. Referring to FIGS. 7-8, FIG. 8 shows four LEDs 132-1 . . . 132-4 emanating from the inner surface 123, however any suitable number of LEDs may be provided based on the intensity and wavelength of the therapeutic light sought for irradiation. Any suitable propagated wavelength of the electromagnetic spectrum may be provided if an illumination element can be so equipped. The underside 108 rests on the dermal surface 10 at the treatment region, adhered by the adhesive member 116 as discussed above. The protrusion 124 has a bottom flush with the underside 128, and opens to define the illumination cavity 118. The lateral extension 128 is flush with the underside 128 for resting on the skin surface, and extends in an articulated manner for protrusion 124 to form the elevated surface 126 at the vessel gap 122.

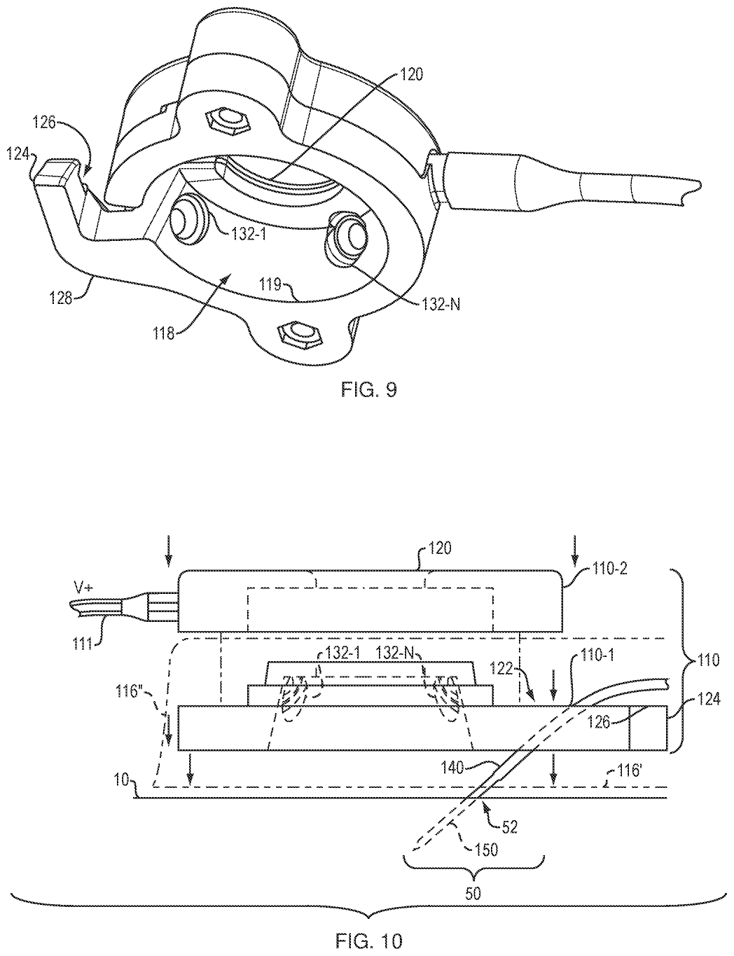

FIG. 9 shows an underside perspective view of the device and illumination cavity 118 of FIGS. 6-8. The illumination cavity 118 is based on a generally concave region under the central gap 120 and extending to an inner perimeter 119 of the circumferential body 110, with the vessel gap 122 allowing passage of the treatment vessel 140.

FIG. 10 shows a method of applying the antimicrobial light treatment of FIGS. 1-9. Referring to FIGS. 1-10, a method for antimicrobial light treatment of a percutaneous insertion site as shown in FIG. 10 includes applying an adhesive member 116 to a treatment region 50 for securing a percutaneous insertion member in an insertion site. The percutaneous insertion member 150, such as a needle, is in fluidic communication with a medication vessel 140 for delivering medication, typically an IV line, infusion line or similar delivery system. The adhesive member 116 may adhere on the epidermal surface, shown as dotted line 116′, or may be applied over the circumferential member 110, shown as dotted line 116″. In the alternate configuration of FIGS. 3A-3B, the adhesive member 116″ may reside between the proximate layer 110-1 and distal layer 110-2.

In either configuration, the circumferential body 110 is disposed onto the treatment region 52. The circumferential body 110 extends generally circular around a central void 120, and placement centers the central void around the insertion site so that the central void allows clearance for the medication vessel 140 and any uninserted portion of the rigid insertion member. The circumferential body 110 may be any suitable shape and size based on the treatment region 50 and the intensity of the illumination source 130 thereby irradiating the treatment region.

The circumferential body 110 includes a discontinuous portion defining the vessel gap 122, which may be continuous with the central void 120. In conjunction with placement of the circumferential body 110, the medication vessel is routed over the elevated surface 126 on the protrusion 124 extending from the circumferential body for permitting the vessel to extending through the vessel gap 122 above and out of contact with the skin surface. The treatment region 50 is illuminated from one or more LEDs (Light Emitting Diodes) 132 disposed on an inner surface of the circumferential body 110 for irradiating an illumination cavity 118 defined by the inner surface and the central void. The LEDs 132 or other illumination source irradiate the treatment region for maintaining an antimicrobial and sterile environment around the insertion site 50. This prevents pathogens from entering the patient along the insertion member 150.

In a particular configuration shown in FIGS. 3A-3C above, the circumferential body has multiple, engageable parts. A first, proximate layer 110-1 accompanies the insertion member 150. Disposing the circumferential body 110 further comprises disposing the proximate layer 110-1 by applying a proximate layer centered on the treatment region using the adhesive member 116, and engaging the distal layer 110-2 onto the proximate layer 116-1 by circumferentially aligning the distal layer with the proximate layer, the LEDs directed towards the illumination cavity. Any suitable adhesive, friction, interference and/or deformable (i.e. snap-fit plastic tab) mechanism may be employed for engaging the proximate 110-1 and distal 110-2 layers.

FIG. 11 shows a perspective view of an alternative configuration of the safety device for use with an epidermal incision and/or treatment. Referring to FIGS. 1 and 11, an antibacterial device 400 includes a polygonal frame 410 having an array of lights 430 around an inner perimeter 418 and directed for focusing the array of lights on a central void 420 for illuminating a epidermal treatment region 450 (FIG. 12) with antibacterial illumination with light of a predetermined wavelength. The predetermined wavelength defines a wavelength having an ability to eradicate microorganisms for preventing infection when the frame is disposed on a skin surface around an incision, infection or other breach in the dermal surface.

In a particular configuration, the illumination source includes one or more light emitting diodes (LEDs) 432, such that each LED provides an antimicrobial and safe light emission in a wavelength around 222 nm or 405 nm. A wavelength in a range between 217-227 nm or 400-410 nm may also provide beneficial results.

The frame 410 may be formed from a light conductive medium formed including one or more of light-transmissive materials such as polymethyl methacrylate (PMMA), silica/quartz, thermoplastic polyurethane (TPU), flexible acrylic, transparent polyvinyl chloride (PVC), UV-inhibitor-free transparent PVC and solar cell material.

FIG. 12 shows a top view of the device of FIG. 11 engaged with an epidermal treatment region. In FIG. 12, the frame 410 defines a central void 420 and a transparent window 421 across the central void 420 for visual observation of an epidermal treatment region 450. The frame 410 is preferably centered over an incision 452 or breach of the epidermal surface. The illumination source disposed on the frame directs the antibacterial light of a predetermined wavelength at the central void 420 for illuminating the epidermal treatment region 450 and incision 452. A size or area of the central void 420 provides illumination of an area adjacent the incision for maintaining a buffer of an irradiated, sterile region around the incision. The array 430 of LEDs 432 defining the illumination source is therefore disposed for irradiation of the dermal region defined by the central void 420. The plurality of LEDs are each arranged to focus towards the central void for providing illuminating coverage of a incision. A slight downward focus towards the dermal surface may also be provided.

In particular configurations, the frame 410 has an adhesion on an underside, similar to the IV irradiation device above, where the adhesion provides temporary attachment to the dermal surface in a region surrounding the epidermal treatment region. Adhesion can also seal the dermal area if the frame 410 is sufficiently flush with the soft tissue in the epidermal treatment region 450, which provides a barrier to further infections influx while the illumination source eradicates the pathogens already in the treatment region defined by the frame 410. The illumination source and/or LEDs generate a light source having a wavelength of 222 nm far UVC or 405 nm visible blue light, or of similar wavelengths having comparable antibacterial effects for eradicating microorganisms.

The wound closure safety device also employs a connection 434 to a power supply for activating the illumination source, or may be powered by on-board batteries. When used as a wound treatment, it is expected that constant usage, such as for inpatient recovery, would be best suited by a hardwired power source.

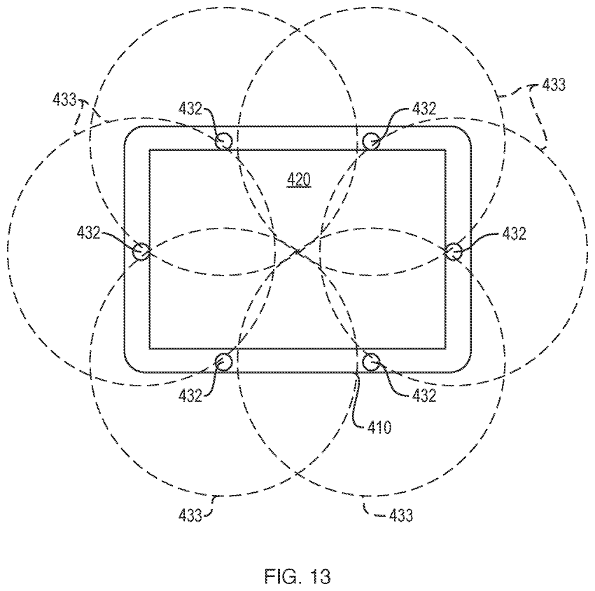

FIG. 13 shows a schematic view of the device of FIGS. 11 and 12 for wound treatment coverage. The LEDs 432 occupy a series of substantially equidistant locations along the inner perimeter 418 of the frame 410. Each of the LEDs 432 has an illumination radius 433 for irradiating the dermal region, so that an aggregation of the illumination radii combined from each respective LED of the plurality or LEDs covers the central void 420. In other words, each position in the central void 420, surrounded by the frame 410, is within an effective treatment radius 433 from at least one of the LEDs 432. Alternatively, the illumination source 430 may form a continuous perimeter around an inner circumference 418, of the frame, where the effective radius 433 would extend at least half the width of the frame for complete coverage.

A corresponding method for deployment of the epidermal device for sterile maintenance of a wound region includes identifying an epidermal treatment region 450 having a therapeutic need for a sterile environment, such as an incision 452 healing. The frame 410 arranges an array of illumination elements of LEDs 432 on the frame for defining an illumination source, where the frame having a shape based on the epidermal treatment region 450. Various sizes of frames 410 may be employed, similar to the manner in which various sizes of bandages are employed for various size skin regions. The frame engages an epidermal surface at the treatment region 450 for directing light of the predetermined therapeutic wavelength at the epidermal treatment region, and energizes the illumination source for irradiating the epidermal treatment region with the light of the therapeutic wavelength.

While the system and methods defined herein have been particularly shown and described with references to embodiments thereof, it will be understood by those skilled in the art that various changes in form and details may be made therein without departing from the scope of the invention encompassed by the appended claims.

What is claimed is:

1. A light-emitting device, comprising:
a frame having a central void, the central void adapted to surround a wound or surgical site; and
an illumination source disposed on the frame for directing safe, therapeutic light centrally towards the underlying wound or surgical site.

2. The device of claim 1 wherein the light has the ability to eradicate microorganisms without harming the patient.

3. The device of claim 1 wherein the light enhances wound healing.

4. The device of claim 1 wherein the illumination source includes one or more light emitting diodes (LEDs), each LED providing an antimicrobial or wound healing wavelength of light.

5. The device of claim 1 wherein the illumination source is disposed for irradiation of a dermal region defined by the central void.

6. The device of claim 1 wherein the illumination source includes a plurality of LEDs arranged towards the central void for providing illuminating coverage of an incision or wound.

7. The device of claim 1 wherein the frame has an underlying adhesive, the adhesion providing temporary attachment to the dermal surface.

8. The underlying adhesive of claim 7 further comprising a transparent, absorptive dressing.

9. The device of claim 1 wherein the light wavelength is far UVC, visible blue, or red light.

10. The device of claim 6 wherein each of the LEDs has an illumination radius for irradiating the dermal region, an aggregation of the illumination radii combined from each respective LED of the plurality or LEDs covers the central void.

11. The device of claim 1 wherein the illumination source forms a continuous perimeter around an inner circumference of the frame.

12. A method including disinfecting and/or enhancing healing of a wound or surgical site, comprising placing a light-emitting device with a frame having a transparent central void, the central void adapted to surround the wound or surgical site;
an illumination source disposed on the frame for directing safe, therapeutic light centrally towards the underlying wound or surgical site; and the transparent central void enabling visualization and clinical monitoring of the underlying wound or surgical site.

13. The method of claim 12 further comprising:
identifying an epidermal treatment region having a therapeutic need for a sterile environment;
arranging an array of illumination elements on a frame for defining an illumination source, the frame having a shape based on the epidermal treatment region;
engaging the frame with an epidermal surface for directing light of a predetermined therapeutic wavelength at the epidermal treatment region; and
energizing the illumination source for irradiating the epidermal treatment region with the light of the therapeutic wavelength.

14. A therapeutic light-emitting device comprising a frame having an array of lights around an inner perimeter and directed for focusing the array of lights on a central void for illuminating an epidermal treatment region with antibacterial or wound-healing illumination.

15. A therapeutic light-emitting device including a centrally positioned light-conductive medium formed from a material selected from the group of light-transmissive materials consisting of polymethyl methacrylate (PMMA), silica/quartz, thermoplastic polyurethane (TPU), flexible acrylic, transparent polyvinyl chloride (PVC), UV-inhibitor-free transparent PVC and solar cell material.
